(19) United States
(12) Patent Application Publication
(10) Pub. No.: US 2003/0170740 A1
(43) Pub. Date: Sep. 11, 2003

(54) Characterized E2IG3 protein for diagnosis and treatment of proliferative diseases

(76) Inventors: Christos Hatzis, Cambridge, MA (US); Nandan Padukone, Melrose, MA (US); John G. Babish, Brooktondale, NY (US)

Correspondence Address: KENYON & KENYON, ONE BROADWAY, NEW YORK, NY, 10004, US

(21) Appl. No.: 10/285,124

(22) Filed: Oct. 31, 2002

Related U.S. Application Data
(60) Provisional application No. 60/335,082, filed on Oct. 31, 2001.

(51) Int. Cl. G01N 33/53; G01N 33/567; C07H 21/04; A61K 39/395; C07K 14/72; A61K 48/00; C07K 16/30

(52) U.S. Cl. 435/7.2; 435/69.1; 435/320.1; 435/325; 424/143.1; 514/44; 530/350; 536/23.5

(57) ABSTRACT

The invention relates to the gene encoding the mammalian E2IG3 and its product. More specifically, the invention relates to the diagnosis of aberrant E2IG3 gene or gene product expression, the identification, production, and use of compounds which modulate E2IG3 expression or the activity of the E2IG3 gene product including but not limited to nucleic acid encoding E2IG3 and homologues, analogues, and deletions thereof, as well as antisense, ribozyme, triplehelix, antibody, and polypeptide molecules as well as small inorganic molecules; and pharmaceutical formulations and routes of administration for such compounds.

### FIELD OF THE INVENTION

The present invention relates to the biological function of a newly identified estrogen-induced protein (E2IG3) and to the use of E2IG3 in the diagnosis, prevention and treatment of proliferative disorders. Additionally, E2IG3 protein may be used as a marker to identify therapeutic or toxic compounds.

### BACKGROUND OF THE INVENTION

With the recent sequencing of the entire human genome and the accumulation of vast amounts of DNA sequences in databases, researchers are realizing that merely having complete sequences of genomes is not sufficient to elucidate biological function or pathology. Information buried in the human genome can be used (1) to identify genes that are central to cellular characteristics in each tissue; (2) to define relationships among genes in specific cellular pathways; (3) to examine genetic motifs on a physiological global scale; (4) to type tumors using expression patterns to complement classical histology and predict disease development; and (5) to monitor the impact of a drug on a pathological state or (6) assess potential toxicological effects of therapeutics.

However, gene expression data are only a portion of the information necessary to accurately characterize cellular changes due to physiological adaptation, pathogenesis or exposure to xenobiotic agents. To fully understand the relationship between a cell and its environment, (1) gene expression profiles must be determined; (2) protein expression and associated post-translational modifications of proteins described; and (3) changes in both gene expression and protein processing must be coordinated. Moreover, the association between gene expression and protein processing must be presented in a manner that allows for rapid identification of the relative involvement and interactions of numerous cellular pathways. At this time, no such process or methodology has been described in the literature.

A cell is normally dependent upon a multitude of metabolic and regulatory pathways for both homeostasis as well as survival. There is no strict linear relationship between gene expression and the protein complement or proteome of a cell.

In cells, the intricate relation between the synthesis of DNA, RNA and protein is circular and can be diagramed as presented in FIG. 1. DNA directs the synthesis of RNA, and RNA then directs the synthesis of protein; special proteins catalyze and regulate the synthesis and degradation of both RNA and DNA. This cyclic flow of information occurs in all cells and has been called the “central dogma” of molecular biology. Proteins are the active working components of the cellular machinery. Whereas DNA stores the information for protein synthesis and RNA carries out the instructions encoded in DNA, proteins carry out most biological activities; their synthesis and ultimate structure are at the heart of cellular function.

Messenger RNA (mRNA) encodes the genetic information copied from DNA in the form of a sequence of nucleotide bases that specifies a sequence of amino acids. The process of expressing the genetic information of DNA in the form of mRNA is termed transcription. On the other hand, translation refers to the whole procedure by which the base sequence of the mRNA s used to order and to join amino acids into a specific linear sequence of a protein; the resulting primary amino acid sequence is the initial determinant of protein structure.

Cellular identity and function are a direct result of both transcriptional and translational control processes. Since all cells possess identical genetic material, transcriptional control is necessary to differentiate one cell type from another. Transcriptional regulatory proteins, a family of DNA-binding proteins, control the expression of genes. Synthesis, processing and stabilization of mRNA by various enzymes and structural proteins represent additional controls to gene expression.

In addition to the variety of transcriptional controls developed by cells, ultimate protein functioning is dependent on several post-translational processes that affect protein structure and hence function. Proteolytic processing is employed to produce finished protein products from primary protein products. Other post-translational modifications include (1) farnsylation, phosphorylation and dephosphorylation; (2) protein-protein interactions to form homo- or heteromeric complexes; and (3) intracellular compartment translocation.

The application of biotechnology to the understanding of gene structure and gene expression is defined as genomics. Currently one of the most active areas in molecular biology, genomics is providing enormous amounts of information regarding the composition of the human genome and transcriptional control. An underlying assumption in genomics is that gene expression as measured by mRNA is an accurate indicator of protein expression and functioning. However, studies on the relationship between mRNA abundance and protein expression have indicated that this association is less than 0.5.

Due to the poor association between transcription and the presence of mature, functional protein, a subset of genomics, termed proteomics, has developed that focuses specifically on the measurement of protein expression in the cell. Methods for measurement of cellular proteins are generally more laborious and have not been modified to provide high-throughput as have methods for the analysis of nucleic acids. Therefore, proteomic research lags far behind genomic research. While high throughput techniques have allowed for the development of data bases concerning transcriptional changes following exposure of cells to exogenous agents, the present state of knowledge as to how any exogenous agent perturbs protein expression and post-translational modification is such that not even experts in the field can estimate what changes will occur.

Proteomic Methods

A cell is normally dependent upon a multitude of metabolic and regulatory pathways for homeostasis and adaptive responses. Since there is no strict linear relationship between gene expression and the protein complement of a cell, both gene and protein expression analyses are necessary to define critical cellular pathways in any biological process. Proteomics is complementary to genomics because it focuses on the gene products, which are the active agents in cells.

Proteomics is the large-scale study of proteins, usually by biochemical methods. The word proteomics has been associated traditionally with displaying a large number of proteins from a given cell line or organism on two-dimensional polyacrylamide gels. However, even when such gels can be run reproducibly between laboratories, determination of the identity of the protein is difficult. In the post-genomic era, protein identification may be affected through a number of laboratory techniques including: (1) one-dimensional gels (with and without affinity purification), (2) two-dimensional gels, (3) micro-chips coated with antibodies, (4) non-denatured protein/protein complexes in solution; (5) post-translational modifiers such as phosphorylation or glycosylation; (6) functional assays for enzyme activity; (7) bioassays for cytokines or receptor/ligand binding; (8) localization of proteins within the cell; (9) large-scale mouse knockouts; (10) RNA interferences; (11) large-scale animal assays for functional proteins; and (12) differential display by two-dimensional gels.

Moreover, academic and commercial interest is moving from the genome to the proteome. There are three significant reasons for this movement. First, automated gene sequencing is reaching maturity as the emphasis expands from de novo sequencing. High-throughput automated DNA sequencing technologies have enabled sequencing of complex genomes. Second, understanding gene expression and protein interactions are likely to be more important than genomics. Researchers want to know what proteins are expressed and to what degree. As previously indicated, DNA expression is literally half the story. Altered protein and peptide expression is generally the key to understanding disease mechanisms. Finally, proteomics will engender a broader range of applications than genomics. In addition to the new areas in academic research and development proteomics will significantly affect drug discovery, preclinical research, clinical research, clinical diagnostics, veterinary medicine, forensics, agrochemical and naturaceuticals. Central to the integration of genomic and proteomic data is the application of sophisticated data handling and bioinformatic techniques to the large data sets characteristic of each methodology.

Information Management

Efforts to characterize the gene expression patterns of the approximately 35,000 human genes are already producing large datasets. According to some estimates, in 3-5 years more than 10⁵ datasets will be available for analysis of the global gene expression patterns of the complete human genome. However, systems capable of analyzing and interpreting data collected from genomic-scale gene expression and proteomic studies are still in their infancy. Such systems will allow comparisons of the expression behavior of individual genes across tissues, developmental and pathological states, or responses to cellular perturbations. To enable these analyses, data warehousing systems are needed to support: (1) data cleaning and verification; (2) integration of data from multiple sources; (3) consistent data models to standardize content of similarly named fields across databases, such as the Gene Expression Markup Language (GEML).

Statistical Methods for Analysis of Genomic Data

The advent of cDNA and oligonucleotide microarray technologies has led to a paradigm shift in biological investigation, such that the bottleneck in research is shifting from data gathering to data analysis. Considering the complexity of the genetic regulatory networks, predictive analysis of the expression patterns is not possible at genome-wide scale. Instead, exploratory analysis methods are typically employed to recognize any non-random patterns or structures in the data, which are then explained based on domain knowledge.

Several exploratory techniques have been recently used to interpret this mass of data. Among the most common, bottom-up hierarchical clustering algorithms use comprehensive pair-wise comparisons to determine similarly expressed genes. Results of these algorithms can be displayed in an intuitive way, but a number of limitations including poor scalability, a tendency to produce a large number of smaller clusters, and lack of global optimization due to the agglomerative nature of the algorithms limit their applicability in the analysis of large, complex datasets. Top-down clustering algorithms, such as K-means clustering, mixture components, and support vector machines, can produce globally optimal cluster structure and also allow the incorporation of prior knowledge to bias the clustering process. However, their application requires specification of number of cluster centers or prior examples to train the algorithms. Finally, projection clustering methods such as principal component analysis, multi-dimensional scaling and self-organizing maps have the advantages of eliminating redundant information and are computationally efficient, but the results could be difficult to interpret if the projection to lower dimensions is not biologically meaningful.

New classes of clustering techniques have been developed specifically for analyzing gene expression data. Of these, gene shaving is optimized for 2-way clustering and can be applied, for example, to find genes that vary the most across conditions. Another promising class of algorithms is the plaid clustering models, which allow for overlapping clusters and memberships in multiple clusters reflecting more realistically the multifunctional nature of many gene products.

Statistical Methods for Analysis of Proteomic Data

Perhaps due to the relatively limited availability of large-scale proteomics datasets, methods for analysis of proteomic patterns are not as well developed. At the exploratory level, the same methods used in gene expression analysis could be employed to detect patterns in proteomic profiles. The regulatory interactions between proteins can then be deduced from the clustering patterns of time-resolved measurements and captured using simple representations of genetic networks based on Boolean models.

An ideal process for the identification of genetic and proteomic pathways involved in homeostasis or pathophysiology would provide information and simultaneous analyses of both gene expression arrays and proteomic changes. Optimally, the procedure should condense the wealth of information generated into summary statistics that are biologically relevant and easy to understand. Furthermore, the process should be applicable to a variety of techniques for the measurement of gene expression arrays as well as protein processing.

### SUMMARY OF THE INVENTION

The present invention provides polypeptides comprising an E2IG3 sequence or a substantially identical fragment thereof, polynucleotides comprising a sequence encoding an E2IG3 polypeptide or substantially identical a fragment thereof, and antibodies that bind to an E2IG3 polypeptide or a substantially identical fragment thereof. These inventive polypeptides, polynucleotides, and antibodies can be used to treat or prevent proliferative diseases in a mammals.

The present invention also provides methods for identification of E2IG3 polypeptide function comprising treating cells with estrogen; determining polynucleotide expression patterns using a gene array; determining the polypeptide phosphorylation expression patterns; and correlating the polynucleotide and polypeptide phosphorylation expression patterns. Method for identification of molecules that modulate E2IG3 expression comprising adding candidate molecules to the culture medium of cells expressing E2IG3 mRNA; measuring E2IG3 expresssion; and comparing the level of E2IG3 expression in the presence of the candidate molecules to the level of E2IG3 expression in the absence of the candidate molecules are further provided by the present invention. Finally, the present invention provides methods for identification of molecules that modulate E2IG3-mediated apoptosis comprising adding candidate molecules to the culture medium of cells expressing E2IG3 mRNA; measuring the degree of apoptosis; and comparing the degree of apoptosis in the presence of the candidate molecules to the degree of apoptosis in the absence of the candidate molecules.

### BRIEF DESCRIPTION OF THE DRAWINGS

FIG. 1 is the genetic code for amino acids and corresponding nucleotide triplet sets.

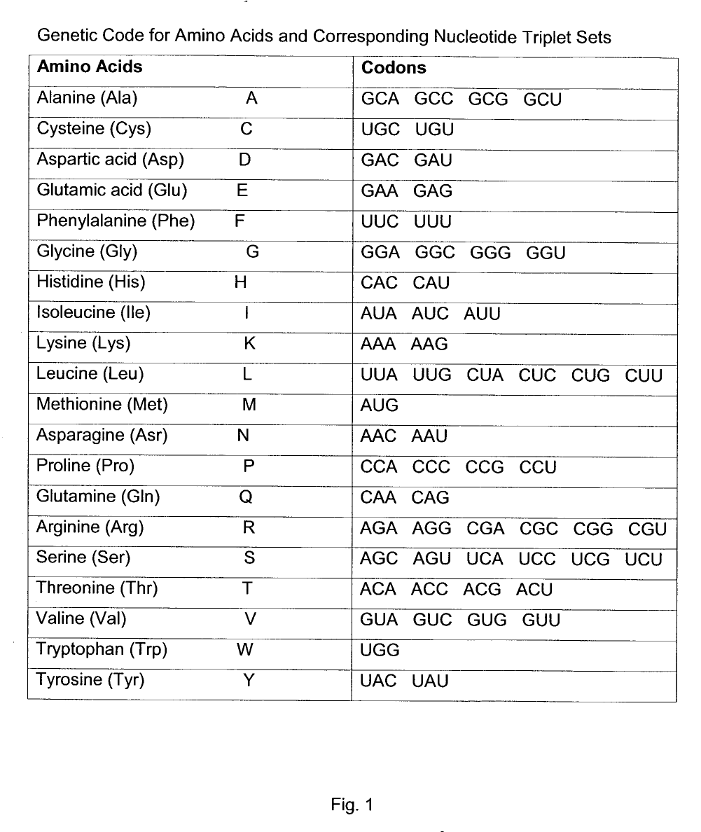

FIG. 2 is the nucleotide sequence of E2IG3.

FIG. 3 is the complete amino acid sequence of E2IG3.

FIG. 4 is an immunoblot of phosphotyrosine proteins in MCF-7 breast cancer cells following treatment with estradiol for 0, 0.25, 3 and 10 hours. Lanes 1, 2 and 3 are solvent controls for 0.25, 3 and 10 hours, while lanes 4, 5 and 6 are the respective estradiol (10 nM) treated cells.

FIG. 5 is an immunoblot of phosphothreonine proteins in MCF-7 breast cancer cells following treatment with estradiol for 0, 0.25, 3 and 10 hours. Lanes 1, 2 and 3 are solvent controls for 0.25, 3 and 10 hours, while lanes 4, 5 and 6 are the respective estradiol (10 nM) treated cells.

FIG. 6 represents the most probable function and pathway involvement of E2IG3 based upon genomic clustering and proteomic analysis.

### DETAILED DESCRIPTION OF THE INVENTION

The present invention is directed to a method of identification of protein function comprising the use of a gene array, composed of several hundred to tens of thousands of genes, capable of discerning the expression of genes within a biological cell.

The term “proliferative disease” describes a disease that is caused by or results in inappropriately high levels of cell division, inappropriately low levels of apoptosis or both. For example, cancers such as breast cancer, ovarian cancer, prostate cancer, lymphoma, leukemia, melanoma, pancreatic cancer and lung cancer are examples of proliferative diseases. Additionally, nonmalignant diseases such as psoriasis, osteoarthritis, inflammatory bowel disease, eczema, Crohn's disease, and acne rosacea are further examples of proliferative diseases.

The term “polypeptide” used herein describes any combination of two or more amino acids, regardless of post-translational modification such as glycosylation of phosphorylation. “E2IG3” or “E2IG3 biological activity” relates to any activity caused in vivo or vitro by E2IG3 or an E2IG3 polypeptide.

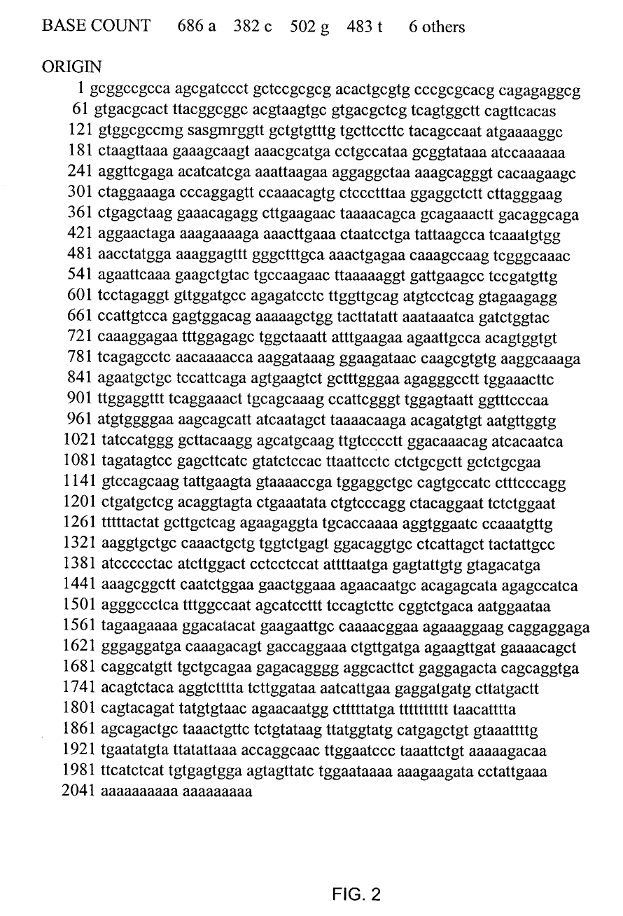

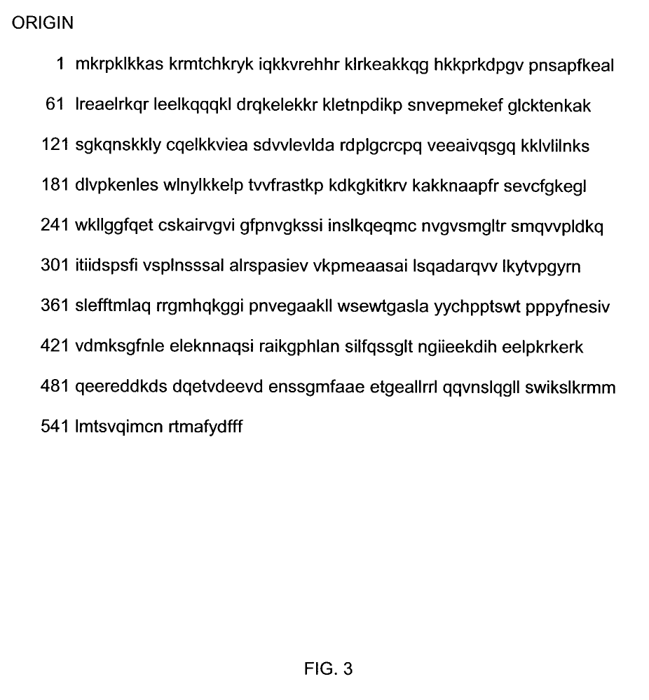

By “substantially identical” is meant a polypeptide or nucleic acid exhibiting at least 50%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at lest 60 nucleotides, more preferably at least 75 nucleotides, and most preferably 110 nucleotides (See FIGS. 2 and 3).

Sequence identity is typically measured using sequence analysis software with the default parameters specified therein (e.g. Sequence Analysis software package of the Genetics Computer Group, University of Wisconsin Biotechnology Center, Madison Wis. 53705). This software program matches similar sequences by assigning degrees of homology to various substitutions, deletions, and other modification. Conservative substitutions typically include substitutions within the following groups: glycine, alanine, valine, isoleucine; aspartic acid, glutamic acid, asparagine, glutamine; serine, threonine; lysine, and phenylalanine, tyrosine.

By “substantially pure polypeptide” is meant a polypeptide that has been separated from the cellular components that naturally accompany it. Typically, the polypeptide is substantially pure when it is at least 60%, by weight, free from the proteins and organic molecules with which it is naturally associated. Preferably, the polypeptide is all E2IG3 polypeptide that is at least 75%, more preferably at least 90%, and most preferably at least 99%, by weight, pure. A substantially pure E2IG3 polypeptide may be obtained, for example, by extraction from a natural source (e.g. a breast cancer cell), by expression of a recombinant nucleic acid encoding an E2IG3 polypeptide or protein, or by chemically synthesizing the protein. Purity can be measured by any appropriate method, e.g. column chromatography, polyacrylamide gel electrophoresis, or HPLC analysis.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

By “substantially pure polynucleotide” is meant DNA that is free of the genes that, in the naturally occurring genome of the organism from which the DNA of the invention is derived, flank the gene. The term therefore include, for example, a recombinant DNA which is incorporated into a vector, into an autonomously replicating plasmid or virus; or into the genomic DNA or a prokaryote or eukaryote; or which exists as a separate molecule (e.g. a cDNA or a genomic or cDNA fragment produced by PCR or restriction endonuclease digestion) independent of other sequences. It also includes a recombinant DNA that is part of a hybrid gene encoding additional polypeptide sequence.

By “transformed cell” is meant a cell into which (or into an ancestor of which) has been introduced, by means of recombinant DNA techniques, a DNA molecule encoding (as used herein) an E2IG3 polypeptide.

By “transgene” is meant any piece of DNA that is inserted by artifice into a cell, and becomes part of the genome of the organism that develops from that cell. Such a transgene may include a gene which is partly or entirely heterologous (i.e. foreign) to the transgenic organism, or may represent a gene homologous to an endogenous gene of the organism.

Ascertaining the function of proteins derived from uncharacterized gene(s) is a fundamental problem in biology. Here we describe by example how the invention can be used to assign a cellular function to a gene with unknown function without addressing gene homology.

However, before the present composition and methods of making and using thereof are disclosed and described, it is to be understood that this invention is not limited to the particular configurations, as process steps, and materials may vary somewhat. It is also intended to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

Agglomerative algorithms such as hierarchical clustering, which are very widely used for analysis of gene expression data, start with each object (gene) being in a separate class. At each step, the algorithm finds the pair of the “most similar” objects, which are then merged in one new class and the process is repeated until all objects are grouped. Agglomerative algorithms produce a very large number of clusters when several thousands objects are involved in the data set.

One common problem with the interpretation of clustered data is to determine the “true” number of clusters. Agglomerative algorithms do not offer explicit “stopping rules” for determining the globally optimal number of classes but rather present the entire set of clusters to the user, who then has to decide on the proper degree of structure in the data.

We have used a partitioning k-means clustering algorithm to cluster the gene expression profiles iteratively into a maximum of 50 classes. This algorithm can produce a globally optimal solution since it starts with the entire data set. At each step of the algorithm the least homogeneous cluster is sub-partitioned and the process is repeated until a criterion for cluster “compactness” is met. Cluster homogeneity, or compactness, is based on the concept of fitness. The later is defined as the sum of distances observations from their corresponding cluster centroid, or

$$Fitness ( C ) = {\sum }_{k = 1}^{C} {\sum }_{i = 1}^{{N}_{k}} d ( {X}_{ik} , \overline{{X}_{k}} ) \qquad ( 1 )$$

where Xᵢₖ is the I-th observation vector assigned to the k-th cluster, Xₖ is the vector of the k-th cluster centroid, Nₖ is the number of observations, or size, of the k-th cluster, C is the number of clusters, and d(x,y) is the distance metric (typically the Euclidian distance) between two vectors. The fitness is largest for C=1 (entire population) and monotonically approaches zero as C approaches N, the total number of observations.

Cluster homogeneity is defined now as:

$$H ( c ) = [ 1 - \frac{Fitness ( c )}{Fitness ( 1 )} ] \times 100 \qquad ( 2 )$$

that takes asymptotically the value of 100%. The optimal number of clusters C*<N is found at a homogeneity level of less than 100, depending on the internal structure of the data.

Thus the present invention is directed to a method for establishing the biological activity of a protein derived from a gene or genes or describing physiological changes by assessing whether structural changes that are induced in proteins present in the eukaryotic cell are coordinate with alterations in gene expression. According to the present invention, any method used to study post-translational changes in cellular proteins can be used to assess whether structural changes have occurred in cellular proteins in response to a test material. For example, such structural changes include protein-protein interactions and protein phosphorylation.

Gene expression analysis may be measured by any of several procedures available in the art. For example, mRNA (˜1 μg) is isolated from the test eukaryotic cells to generate first-strand cDNA by using a T7-linked oligo(dT)primer. After second-strand synthesis, in vitro transcription (Ambion) is performed with biotinylated UTP and CTP (Enzo Diagnostics), the result is a 40- to 80-fold linear amplification of RNA. Forty micrograms of biotinylated RNA is fragmented to 50- to 150-nt size before overnight hybridization to Affymetrix (Santa Clara, Calif.) HU6000 arrays. Arrays contain probe sets for 6,416 human genes (5,223 known genes and 1,193 expressed sequence tags (EST)). Because probe sets for some genes are present more than once on the array, the total number on the array is 7,227. After washing, arrays are stained with streptavidin-phycoerythrin (Molecular Probes) and scanned on a Hewlett Packard scanner. Intensity values are scaled such that overall intensity for each chip of the same type is equivalent. Intensity for each feature of the array is captured using the GENECHIP SOFTWARE (Affymetrix, Santa Clara, Calif.), and a single raw expression level for each gene is derived from the 20 probe pairs representing each gene by using a trimmed mean algorithm. A threshold of 20 units is assigned to any gene with a calculated expression level below 20, because discrimination of expression below this level is not performed with confidence in this procedure. Alternatively, the SAGE system for gene expression analysis may be used.

The following examples are intended to illustrate, but not in any way limit, the invention. Detailed descriptions of conventional methods, such as those employed herein, can be obtained from numerous publications, including, for example, Sambrook et al., Molecular Cloning: A Laboratory Manual, 2^{nd} ed., Cold Spring Harbor Laboratory Press (1989). All references mentioned herein are incorporated in their entirety.

### EXAMPLES

### Example 1

### Delineating Function of the Protein Product of E2IG2

Summary

The present example demonstrates the identification of protein function using a gene array composed of several hundred genes to tens of thousands of genes. Specifically, the putative function of a novel estrogen-induced protein (E2IG3 or G3) was determined to be involved in protein kinase pathways linking cell surface receptors with cell growth and maintenance. In particular, G3 was determined to be involved in MAPKinase and cell cycle signal transduction pathways, as well as in protein amino acid phosphorylation in signaling pathways involving I-kappaB. Furthermore, G3 was determined to be a putative GTPase with tyrosine kinase activity, associated with membrane kinases linked with the microtubule cytoskeleton or mitochondria. Thus, according to the present example, the composite functional description of G3 is that of a membrane associated or related kinase involved in signaling pathways related to cell growth or maintenance. Also, the putative role of G3 relative to E2 and breast cancer cells appears to be in protection of the cell from apoptosis following estrogen stimulation.

Methods

Equipment

The following equipment used for experiments in this Example includes an Ohaus Explorer analytical balance, (Ohaus Model #EO1140, Switzerland), biosafety cabinet (Forma Model #F 1214, Marietta, Ohio), pipettor, 100 to 1000 μL (VWR Catalog #4000-208, Rochester, N.Y.), cell hand tally counter (VWR Catalog #23609-102, Rochester, N.Y.), CO₂ Incubator (Forma Model #F3210, Marietta, Ohio), hemacytometer (Hausser Model #1492, Horsham, Pa.), inverted microscope (Leica Model #DM IL, Wetzlar, Germany), pipet aid (VWR Catalog #53498-103, Rochester, N.Y.), pipettor, 0.5 to 10 μL (VWR Catalog #4000-200, Rochester, N.Y.), pipettor, 100 to 1000 μL (VWR Catalog #4000-208, Rochester, N.Y.), pipettor, 2 to 20 μL (VWR Catalog #4000-202, Rochester, N.Y.), pipettor, 20 to 200 μL (VWR Catalog #4000-204, Rochester, N.Y.), PURELAB Plus Water Polishing System (U.S. Filter, Lowell, Mass.), Refrigerator, 4° C. (Forma Model #F3775, Marietta, Ohio), vortex mixer (VWR Catalog #33994-306, Rochester, N.Y.), a water bath (Shel Lab Model #1203, Cornelius, Oreg.), microfuge tubes, 1.7 mL (VWR Catalogue #20172-698, Rochester, N.Y.), pipet tips for 0.5 to 10 μL pipettor (VWR Catalogue #53509-138, Rochester, N.Y.), pipet tips for 100- 1000 μL pipettor (VWR Catalogue #53512-294, Rochester, N.Y.), pipet tips for 2-20 μL and 20-200 μL pipettors (VWR Catalogue #53512-260, Rochester, N.Y., pipets, 10 mL (Becton Dickinson Catalog #7551, Marietta, Ohio), pipets, 2 mL (Becton Dickinson Catalog #7507, Marietta, Ohio, pipets, 5 mL (Becton Dickinson Catalog #7543, Marietta, Ohio) and a cell scraper (Corning Catalog #3008, Corning, N.Y.). Equipment for SDS-PAGE includes a Vertical Gel System (Savant Holbrook, N.Y.) and power supply (Savant Instruments Model #PS250, Holbrook, N.Y.).

Chemicals

Chemicals, reagents and buffers necessary include dimethylsulfoxide (DMSO) (VWR Catalog #5507, Rochester, N.Y.), Modification of Eagle's Medium (DMEM) (Mediatech Catalog #10-013-CV, Herndon, Va.), fetal bovine serum, Heat Inactivated (FBS-HI) (Mediatech Catalog #35-011-CV, Herndon, Va.), Penicillin/Streptomycin (Mediatech Catalog #30-001-CT, Herndon, Va.), tissue culture plate, 24-well, 3.4 mL capacity (Becton Dickinson Catalog #3226, Franklin Lanes, N.J.) and ultra-pure water (Resistance=18 megaOhm×cm deionized water). Supplies and reagents for western blotting are 10-20% precast gradient mini-gels (BioWhittaker Molecular Applications Catalog #58506, Rockland, Me.), 2× sample buffer (Sigma Catalog #L-2284, St. Louis, Mo.), beaker, 1000 mL (VWR Catalog #13910-289, Rochester, N.Y.), color molecular weight standard (Sigma Catalog #C-3437, St. Louis, Mo.), glycine (Sigma Catalog #G-7403, St. Louis, Mo.), graduated cylinder, 1000 mL (VWR Catalog #24711-364, Rochester, N.Y.), microfuge tubes, 0.5 mL Safe-Lock (Brinkmann Catalog #22 36 365-4, Westbury, N.Y.), microfuge tubes, 1.7 mL (VWR Catalog #20172-698, Rochester, N.Y.), pipet tips for 2-20 μL and 20-200 μL pipettors (VWR Catalogue #53512-260, Rochester, N.Y.), pipet tips, gel loading (VWR Catalog #53509-018, Rochester, N.Y.), sodium dodecyl sulfate (SDS) (Sigma Catalog #L-4509, St. Louis, Mo.), Stir Bar, Magnetic (VWR Catalog #58948-193, Rochester, N.Y.), storage bottle, 1000 mL (Coming Catalog #1395-1L, Corning, N.Y.), and trizma Base (Sigma Catalog #T-6066, St. Louis, Mo.).

Cell Culture

The human MCF-7 breast cancer cell line (ATCC) was treated with 10 nM 17B-estridiol (E2) and gene and protein phosphorylation expression patterns (tyrosine and threonine) were determined at 0.25, 3 and 10 hours post-exposure. Since gene expression was not expected to be altered due to estrogen receptor regulation at 0.25 hour, only phosphoprotein expression patterns were determined at 0.25 hours. It is expected that signaling patterns derived from E2 interaction with the ER would alter phosphorylation status of proteins at the early time point.

Gene Expression—To obtain mRNA samples for gene expression analysis, MCF-7 cells were seeded into 150-mm plates at 1.5×10⁶ cells per plate and allowed to reach a logarithmic growth phase. At 40% confluency, cells will be incubated for 48 h in culture media and then treated with 10⁻⁸, 10⁻⁷, 10⁻⁶ E2 or vehicle control (equivalent amounts of ethanol). Cells are lysed and samples for mRNA analysis and western blotting for phosphotyrosyl proteins collected at 0, 0.25, 0.5, 2, 4 and 10 h post-treatment. Approximately 1 μg of mRNA from the test eukaryotic cells is used to generate first-strand cDNA by using a T7-linked oligo(dT)primer. After second-strand synthesis, in vitro transcription (Ambion) is performed with biotinylated UTP and CTP (Enzo Diagnostics), the result is a 40- to 80-fold linear amplification of RNA. Forty micrograms of biotinylated RNA is fragmented to 50- to 150-nt size before overnight hybridization to Affymetrix (Santa Clara, Calif.) HU6000 arrays. Arrays contain probe sets for 6,416 human genes (5,223 known genes and 1,193 expressed sequence tags (EST)). Because probe sets for some genes are present more than once on the array, the total number on the array is 7,227.

After washing, arrays are stained with streptavidin-phycoerythrin and scanned. Intensity values are scaled such that overall intensity for each chip is equivalent. Intensity for each feature of the array is captured using GENECHIP SOFTWARE (Affymetrix, Santa Clara, Calif.), and a single raw expression level for each gene is derived from the 20 probe pairs representing each gene by using a trimmed mean algorithm. A threshold of 20 units is assigned to any gene with a calculated expression level below 20, because discrimination of expression below this level is not performed with confidence in this procedure.

Protein quantification was determined from cell lysates using a Packard FluoroCount Model #BF10000 fluorometer (Meriden, Conn.). Other equipment not previously listed included a Forma Model #F3797 −30° C. freezer, Heating Block (VWR Catalog #13259-030, Rochester, N.Y.), Microfuge (Forma Model #F3590, Marietta, Ohio). The procedure described in the NanoOrange Protein Quantiation Kit (Molecular Probes Catalog #N-6666, Eugene, Oreg.) is followed without modification.

Immunoblotting of Phosphoproteins

Prepare 5× SDS-PAGE buffer by dissolving 15 grams of Tris base, 72 grams glycine, and 5 grams SDS in 900 mL distilled water in a 1000 mL beaker with a magnetic stir bar. Place on a magnetic stirrer and stir until dissolved. Adjust volume to 1000 mL with a 1000 mL cylinder. Store at 4° C. Prepare 1× SDS-PAGE buffer by combining 200 mL of the 5× stock with 800 mL water. Store in a 1000 mL storage bottle at 4° C. Warm to room temperature before use. Melt the 2× Sample Buffer at room temperature and store as 500 μL aliquots in 1.7 mL microfuge tubes in −30° C. freezer. Assemble vertical gel system according to manufacturer's guidelines. Pour enough 1× SDS-PAGE buffer into gel system to cover top of gel and enough in bottom of the apparatus to cover bottom of glass plates. Remove a tube of 2× Sample Buffer from freezer and melt at room temperature. Melt frozen cell lysate samples on ice. Dilute cell lysate samples samples 1:1 with 2× sample buffer in 0.5 mL Safe-Lock tubes (15 μL of cell lysate sample and 15 μL 2× Buffer). Put remaining 2× Sample Buffer back into freezer (−30° C.). Put cell lysate samples back into freezer (−80° C.). Heat protein samples and molecular weight standards (if required) to 95-100° C. for 5 minutes. Briefly, spin in microfuge to collect sample at bottom of tube, and load equal amounts of protein in wells of pre-cast gel. Run at 30 mA per gel at constant current for 60 minutes, or until dye reaches the bottom of gel.

Supplies and reagents for western blotting of phosphotyrosyl proteins includes anti-phosphotyrosine and anti-phosphothreonine antibody 4G10 (UBI Lake Placid, N.Y.), blotting Paper (VWR Catalog #28303-104, Rochester, N.Y.), glycine (Sigma Catalog #G-7403, St. Louis, Mo.), hydrochloric acid (HCl) (VWR Catalog #VW3110-3, Rochester, N.Y.), methanol (VWR Catalog #VW4300-3, Rochester, N.Y.), NaOH (Sigma Catalog #S-5881, St. Louis, Mo.), nitrocellulose membrane (Schleicher & Schuell Catalog #10402680, Keene, N.H.), Nonfat dry milk (Carnation Brand), peroxidase labeled goat anti-mouse IgG (KPL Catalog #474-1806, Gaithersburg, Md.), and phosphate buffered saline (PBS) (Mediatech Catalog #21-040-CV, Herndon, Va.).

SDS-polyacrylamide gel electrophoresis for phosphoproteins was performed on using the MCF-7 cell lysate. The membrane was removed from glass plates and equilibrated in Towbin buffer for 5 minutes with gentle rotation at room temperature. Cut nitrocellulose membrane to correct size, nicking off the lower right hand corner. Prewet membrane with ultra-pure water, then equilibrate for 5 minutes in transfer buffer. Prewet 6 pieces of blotting paper for each gel to be transferred in 1× Towbin buffer.

Set up transfer sandwich according to the manufacturer's directions. Transfer proteins at 96 mA per gel for 60 minutes per gel. Check for good protein transfer by staining with 10 mL Ponceau S solution for 5 minutes, then washing several times with water. Block the blotted membrane with 10 mL of freshly prepared PBS containing 3% nonfat dry milk (PBS-NFDM) for 20 minutes at room temperature with constant agitation. Incubate the membrane with the primary antibody diluted to 1 μg/mL in 5 mL freshly prepared PBS-NFDM overnight at 4oC and sealed in a plastic bag.

Wash the membrane twice with water. Incubate the membrane in the secondary antibody diluted 1:3000 in 10 mL freshly prepared PBS-NFDM for 1.5 hours at room temperature with constant agitation. Wash the membrane twice with water. Wash the membrane in PBS-0.05% Tween 20 for 3.5 minutes at room temperature with constant agitation. Wash membrane 3-4 times with water. Detect tyrosine phosphoproteins using chemiluminescence.

Chemiluminescence for visualization of phosphotyrosine proteins was performed using a UVP darkroom with cooled integrated camera (Epi Chemi II Darkroom with LabWorks Software, UVP, Upland, Calif.), LumiGlo® Chemiluminescent Substrates A and B (KPL Catalog #54-61-02, Gaithersburg, Md.). Remove LumiGlo® Chemiluminescent Substrates A and B from refrigerator. After proteins have been blotted to nitrocellulose or PVDV, drain excess water from membrane by touching edge of membrane on a clean KimWipe. Place membrane into a clean weigh boat or other suitable container. Add 0.8 mL of Substrate A and of Substrate B directly to membrane and swirl around to mix. Put LumiGlo® Chemiluminescent Substrates A and B back into refrigerator. Allow substrate to incubate on membrane for 1 minute at room temperature. Remove membrane from weigh boat, drain off excess substrates, and place directly onto the transilluminator of the Epi Chemi II system. In the LabWorks program provided, select On-Chip Integration and integrate for various times until a good signal is achieved (1,3,6,10 and/or 15 minutes, depending on how much protein of interest is present on membrane). Images of phosphotyrosylated proteins were captured on TIFF files for density measurements using Scan Analysis software (Biosoft, Stapleford, Cambridge, UK).

Clustering of Genome-Wide Expression Data

The genome-wide expression pattern was generated from the MCF-7 cell line in the presence of estrogen using the SAGE method (Carpentier et al., Cancer Research, 60, 5977-5983, Nov. 1, 2000). The dataset was obtained from the publicly accessible NCBI SAGE web site. Approximately 61,000 10-bp tags were sequenced for each of the three time points (0 h, 3 h and 10 h). To account for errors inherent in the sequencing of the SAGE tags, tags having fewer than two counts for all three time points were removed from the dataset, as these tags could represent erroneous sequences. Then, using NCBI's tag to gene mapping tables, tags that encode for the same gene transcript were grouped together and averaged to produce the transcription level of the corresponding gene. Finally, transcription level of all genes was expressed as the logarithm-transformed ratio relative to the transcription level of 10 housekeeping genes at each corresponding time point (a count of 1 was added to genes with 0 counts to allow for the logarithmic transformation).

Relating Proteomic Signaling Pathways and Gene Clusters

In order to quantify the extent to which various signaling pathways were affected by E2 and their relationship to the gene clusters formed, a database containing information as to the phosphorylation status and molecular weight of proteins was constructed. The immunoblotting data of relative phosphoprotein expression were then matched to phosphorylation nodes on signaling pathways to produce a score for each pathway. The top signaling pathways in terms of percentage of node hits relative to potential node hits were selected and used to identify annotated gene clusters that included genes whose protein products functioned in the selected proteomic pathway.

Results

The gene clustering process resulted in a set of 7907 genes and the expression levels of these genes were then clustered using the iterative k-means algorithm to characterize the detailed patterns. Table 1.1 describes the 31 clusters that were identified as optimal subsets of the active expression data from the genome-wide experiment.

TABLE 1.1
Clusters of genes optimized as subgroups distinct from overall population

| Cluster | Size | Avg_Log_0h | Avg_Log_3h | Avg_Log_10h | Remarks |
|---|---|---|---|---|---|
| Population | 7905 | 0.32 | 0.38 | 0.42 | |
| 1 | 701 | 0.16 | −0.13 | −0.13 | Down-regulated |
| 2 | 266 | 0.18 | −0.17 | 0.22 | Early Down-regulated |
| 3 | 198 | 0.81 | 0.84 | 0.98 | Up−regulated |
| 4 | 422 | 1.69 | 1.69 | 1.66 | Up−regulated |
| 5 | 375 | −0.21 | 0.35 | −0.13 | Early Up-regulated |
| 6 | 764 | −0.21 | 0.00 | −0.13 | Inactive |
| 7 | 701 | −0.22 | −0.19 | 0.29 | Late up-regulated |
| 8 | 261 | 1.43 | 1.39 | 1.40 | Inactive |
| 9 | 454 | −0.20 | 0.16 | 0.22 | Up-regulated |
| 10 | 182 | 0.15 | 0.37 | −0.12 | Early Up-regulated |
| 11 | 162 | 1.09 | 1.01 | 0.97 | Inactive |
| 12 | 312 | 0.14 | 0.19 | 0.22 | Inactive |
| 13 | 206 | 0.87 | 0.69 | 0.72 | Inactive |
| 14 | 113 | 0.39 | −0.18 | 0.50 | Early Down-regulated |
| 15 | 214 | −0.13 | 0.16 | 0.54 | Up-regulated |
| 16 | 199 | 0.52 | 0.55 | 0.80 | Late up-regulated |
| 17 | 229 | 0.42 | 0.40 | 0.24 | Inactive |
| 18 | 216 | 0.45 | 0.43 | 0.52 | Inactive |
| 19 | 162 | 0.22 | 0.64 | 0.49 | Up-regulated |
| 20 | 197 | −0.10 | 0.45 | 0.24 | Early Up-regulated |
| 21 | 180 | 0.39 | 0.03 | −0.11 | Down-regulated |
| 22 | 98 | 0.51 | 0.43 | −0.13 | Late Down-regulated |
| 23 | 177 | 0.58 | 0.66 | 0.42 | Inactive |
| 24 | 187 | 0.15 | 0.36 | 0.58 | Early Up-regulated |
| 25 | 122 | −0.19 | 0.57 | 0.61 | Up-regulated |
| 26 | 148 | 0.49 | 0.83 | 0.80 | Up-regulated |
| 27 | 169 | 0.37 | 0.12 | 0.35 | Inactive |
| 28 | 117 | 0.49 | 0.17 | 0.71 | Early Down-regulated |
| 29 | 75 | 2.14 | 2.11 | 1.92 | Inactive |
| 30 | 101 | 0.75 | 0.40 | 0.57 | Early Down-regulated |
| 31 | 197 | 1.10 | 1.15 | 1.23 | Inactive |

Cluster 20 is highlighted because it contains the E2IG3 (G3) gene, whose expression is regulated by E2 and whose function is unknown.¹ Clearly, its presence within the early up-regulated genes due to its expression profile suggests behavioral similarities, at the first level, with other genes with similar expression characteristics. The cluster contains ¹ The nucleic acid sequence of the E2IG3 (G3) gene is shown in FIG. 2 and the complete amino acid sequence of E2IG3 (G3)is shown in FIG. 3. 197 genes in total. Using this cluster as a basis and the unknown gene of interest in it, further analyses can be used to assign a relatively specific function to G3.

Analyses of Functional Behavior Within Cluster

Gene Ontology (GO) was developed to provide a standard set of terms for biological processes, molecular function and cellular categories (http://www.geneontology.org). This hierarchically organized ontology has been mapped into the resulted gene clusters to provide automated annotation of each cluster by matching of genes within a cluster to its known functions. Using this mapping technique, cluster 20 genes were fully characterized for functionality. The three ontologies described in GO were independently used: Biological Process, Molecular Function and Localization. Each of these has multiple levels of hierarchy within them that describe function at aggregate to highly detailed levels.

Starting with the highest layer of the GO category, the algorithm calculates the probability of predicting the function in the total population as well as that in cluster 20 based on the frequency of genes with that function in the respective set of genes. The ratio of these two probabilities (cluster 20/population) then provides a predictive score for each function within cluster 20. A score greater than I represents a stronger presence of a function in cluster 20 than in the population as a whole. Statistical tests such as the chi-square test or Fisher's exact test are then applied to associate a statistical significance level to the estimated score. Only those functional categories that meet the significance criterion are retained (typically a 95% significance level is used). Clearly, high scores based on a sizeable gene count represent the strongest functional characterization for the cluster. The results are shown in Tables 1.2 to 1.4.

TABLE 1.2
Biological Process description for cluster 20 genes

| Gene Ontology: | Cluster | Total | |
|---|---|---|---|
| Biological Process | 20 | Population | Score* |
| behavior | 1 | 9 | |
| cell communication | 25 | 619 | 1.62 |
| cell adhesion | 2 | 77 | |
| cell-cell signaling | 1 | 79 | |
| response to external stimulus | 5 | 173 | |
| signal transduction | 18 | 405 | 1.78 |
| cell surface receptor | 5 | 116 | |
| linked signal transduction 3 | 29 | 4.15 | |
| enzyme linked receptor | | | |
| protein signaling pathway | 3 | 32 | 3.76 |
| intracellular signaling cascade | 4 | 78 | |
| protein kinase cascade | 3 | 32 | 3.76 |
| small GTPase mediated | 1 | 30 | |
| signal transduction | | | |
| cell growth and/or maintenance | 53 | 1670 | 1.27 |
| cell cycle | 5 | 210 | |
| cell organization and biogenesis | 3 | 55 | |
| cell proliferation | 5 | 165 | |
| cell shape and cell size control | 2 | 59 | |
| metabolism | 35 | 1144 | |
| carbohydrate metabolism | 1 | 53 | |
| catabolism | 3 | 92 | |
| electron transport | 1 | 25 | |
| energy pathways | 1 | 66 | |
| lipid metabolism | 3 | 88 | |
| nucleobase, nucleoside, | 11 | 471 | |
| nucleotide and nucleic acid | | | |
| oxygen and radical metabolism | 1 | 2 | 20.06 |
| phosphate metabolism | 3 | 23 | 5.23 |
| protein metabolism and modification | 11 | 315 | |
| protein biosynthesis | 2 | 111 | |
| protein complex assembly | 1 | 40 | |
| protein folding | 1 | 28 | |
| protein modification | 7 | 122 | 2.30 |
| protein amino acid phosphorylation | 6 | 62 | 3.88 |
| phosphorylation of I-kappaB | 1 | 1 | 40.13 |
| protein targeting | 1 | 39 | |
| stress response | 3 | 104 | |
| transport | 9 | 200 | |
| death | 5 | 101 | |
| developmental processes | 9 | 282 | |
| physiological processes | 5 | 90 | |

*Scores significant at the 95% level according to a chi-square test are shown

Based upon those Biological Process descriptions elevated in cluster 20 (p<0.05), G3 would be expected to be involved in protein kinase pathways linking cell surface receptors with cell growth and maintenance. Further, G3 is likely (p<0.05) a kinase involved in protein amino acid phosphorylation in signaling pathways involving I-kappaB (p<0.05). Statistically significant Molecular Functions as listed in Table 1.3 support and extend this inference to a putative GTPase with tyrosine kinase activity.

TABLE 1.3
Molecular Function description for cluster 20 genes

| Gene Ontology: | Cluster | | |
|---|---|---|---|
| Molecular Function | 20 | Totals | Score* |
| enzyme | 29 | 849 | |
| hydrolase | 9 | 334 | |
| hydrolase, acting on acid anhydrides | 6 | 148 | |
| hydrolase, acting on acid anhydrides, | 5 | 116 | |
| in phosphorus | | | |
| GTPase | 5 | 54 | 3.7 |
| kinase | 7 | 143 | |
| protein kinase | 5 | 125 | |
| protein tyrosine kinase | 3 | 21 | 5.72 |
| oxidoreductase | 5 | 117 | |
| transferase | 5 | 126 | |
| ligand binding or carrier | 16 | 479 | |
| nucleotide binding | 3 | 40 | 3 |
| purine nucleotide binding | 3 | 36 | 3.33 |
| protein binding | 9 | 281 | |
| transcription factor binding | 4 | 115 | |
| transcription co-factor | 4 | 110 | |
| transcription co-repressor | 3 | 47 | |
| nucleic acid binding | 16 | 565 | |
| DNA binding | 11 | 330 | |
| transcription factor | 8 | 211 | |
| RNA polymerase II transcription factor | 4 | 97 | |
| RNA binding | 5 | 204 | |
| signal transducer | 13 | 284 | 1.83 |
| receptor | 5 | 128 | |
| transmembrane receptor | 3 | 91 | |
| structural protein | 5 | 163 | |
| transporter | 5 | 126 | |

*Scores significant at the 950/0 level according to a chi-square test are shown

TABLE 1.4
Localization description for cluster 20 genes

| Gene Ontology: | Cluster | | |
|---|---|---|---|
| Cellular Component | 20 | Totals | Score* |
| cell | 55 | 1659 | 1.33 |
| cell fraction | 7 | 220 | |
| membrane fraction | 4 | 163 | |
| soluble fraction | 3 | 61 | |
| intracellular | 35 | 1199 | |
| cytoplasm | 23 | 751 | |
| cytoskeleton | 6 | 120 | |
| microtubule cytoskeleton | 3 | 25 | 4.8 |
| cytosol | 4 | 107 | |
| mitochondrion | 5 | 104 | |
| nucleus | 12 | 513 | |
| membrane | 21 | 541 | 1.55 |
| integral membrane protein | 9 | 327 | |
| integral plasma membrane protein | 9 | 254 | |
| mitochondrial membrane | 5 | 73 | 2.74 |
| mitochondrial inner membrane | 3 | 59 | |
| plasma membrane | 7 | 175 | |
| peripheral plasma membrane protein | 3 | 47 | |

*Scores significant at the 95% level according to a chi-square test are shown

Based on localization description for cluster 20 (Table 1.4), G3 is likely (p<0.05) associated with membrane kinases linked with the microtubule cytoskeleton or mitochondria. Thus, the composite functional description of G3 is that of a membrane associated or related kinase involved in signaling pathways related to cell growth or maintenance. An involvement in the phosphorylation of I-kappaB is also implied in the cluster analysis.

Correlating Genomic Clustering with Proteomic Measurements

The function of G3 may be further described by correlating the proteomic results with those of gene clustering.

TABLE 1.5
Protein tyrosine phosphorylation induced by
E2 treatment of MCF-7 cells.

| Molecular Weight | | | |
|---|---|---|---|
| [Da] | T = 0.25 hours | T = 3 hours | T = 10 hours |
| 104,112 | 0.68 | 1.59 | 1.80 |
| 94,842 | 0.91 | 1.24 | 3.64 |
| 86,218 | 1.09 | 2.49 | 1.86 |
| 92,704 | 1.17 | 7.23 | 1.73 |
| 76,692 | 1.00 | 1.89 | 2.80 |
| 70,226 | 0.85 | 1.20 | 1.92 |
| 61,695 | 0.99 | 1.27 | 1.12 |
| 56,964 | 0.92 | 0.91 | 1.26 |
| 53,920 | 0.81 | 0.80 | 1.31 |
| 51,731 | 0.69 | 0.64 | 1.14 |
| 49,785 | 0.72 | 0.39 | 0.86 |
| 46,639 | 1.57 | 0.47 | 0.81 |
| 44,468 | 1.51 | 0.76 | 0.94 |
| 41,831 | 1.10 | 0.97 | 1.09 |
| 40,133 | 0.94 | 0.99 | 1.10 |
| 37,831 | 0.81 | 0.85 | 0.97 |
| 35,958 | 0.57 | 0.73 | 0.79 |
| 34,037 | 0.61 | 0.75 | 0.81 |
| 32,085 | 0.83 | 0.79 | 0.77 |
| 30,339 | 0.67 | 0.70 | 0.64 |
| 28,129 | 0.36 | 0.59 | 0.75 |
| 26,461 | 0.34 | 0.46 | 0.76 |
| 24,840 | 0.43 | 0.36 | 0.66 |
| 22.935 | 0.45 | 0.31 | 0.69 |
| 20,233 | 0.45 | 0.40 | 0.58 |
| 18,974 | 0.68 | 0.36 | 0.55 |
| 17,142 | 0.77 | 0.66 | 0.44 |

TABLE 1.6
Phosphorylation of threonine induced by E2 treatment of MCF-7 cells

| Molecular Weight | | | |
|---|---|---|---|
| [Da] | T = 0.25 hours | T = 3 hours | T = 10 hours |
| 115,213 | 0.66 | 0.64 | 1.13 |
| 107,041 | 0.68 | 0.77 | 1.15 |
| 97,611 | 1.10 | 0.88 | 0.99 |
| 88,920 | 1.11 | 0.87 | 0.76 |
| 79,177 | 0.86 | 0.61 | 0.78 |
| 72,277 | 0.98 | 0.67 | 0.86 |
| 68,415 | 1.05 | 0.59 | 0.64 |
| 64,491 | 1.02 | 1.04 | 0.68 |
| 58,749 | 0.82 | 1.02 | 0.22 |
| 53,573 | 1.02 | 0.88 | 0.77 |
| 47,605 | 6.29 | 1.02 | 0.98 |
| 46,291 | 1.00 | 0.95 | 0.93 |
| 43,053 | 0.96 | 0.73 | 1.16 |
| 39,260 | 0.87 | 0.55 | 0.85 |
| 35,176 | 1.14 | 0.68 | 0.70 |
| 32,885 | 1.09 | 0.74 | 0.58 |
| 29,070 | 1.21 | 0.42 | 2.19 |
| 27,290 | 1.07 | 0.86 | 1.05 |
| 23,605 | 1.57 | 0.03 | 0.73 |
| 21,040 | 1.13 | 0.14 | 0.68 |
| 19,588 | 1.14 | 0.24 | 0.38 |
| 17,679 | 0.37 | 0.57 | 0.50 |

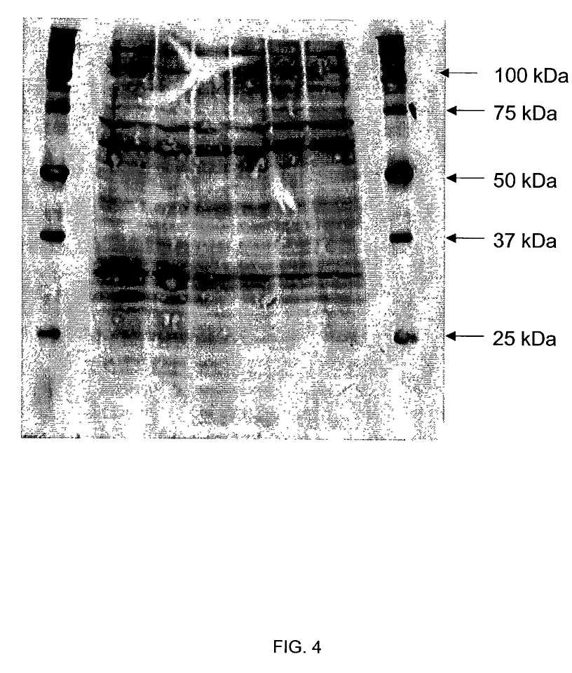

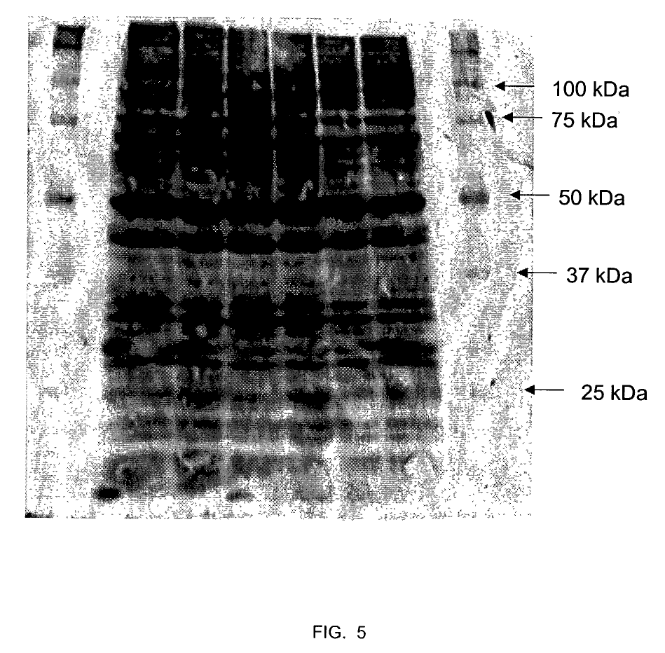

Tables 1.5 and 1.6 describe the relative changes in phosphorylation of tyrosine and threonine, respectively, in the MCF-7 cells 0.25, 3 and 10 hours following treatment with 10 nM E2. They were produced from the immunoblots represented in FIGS. 4 and 5. The gray regions indicate those relative changes greater than 20 percent. When phosphorylation nodes among the signaling pathways represented in the database were compared to the experimental results in Tables 1.5 and 1.6, the highest scoring pathways were the MAPKinase and cell cycle pathways. Scores for MAPKinase tended to be highest at the 0.25 hour time point and decrease at 3 and 10 hours, while cell cycle scores were highest at 3 hours. A slight decrease was noted for cell cycle scores at 10 hours.

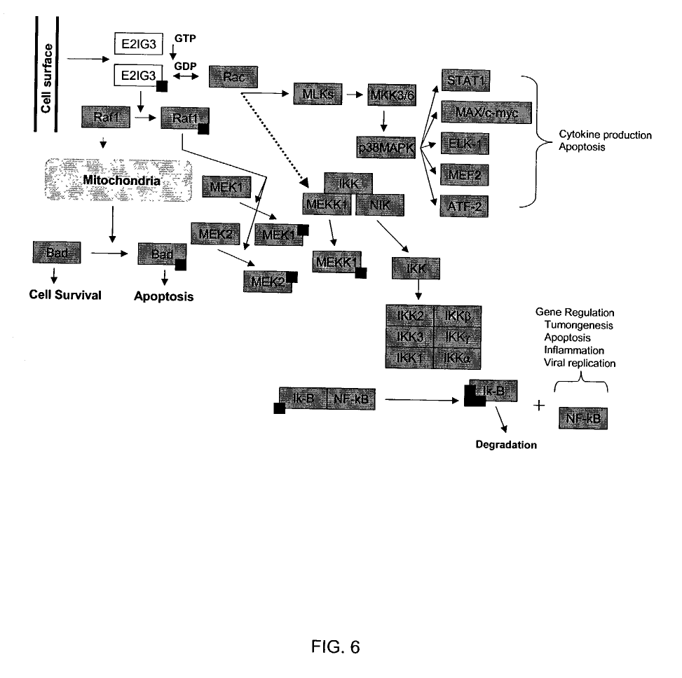

The high degree of association with phosphorylation exhibited by cluster 20, and by inference G3, provided a link to the pathways identified through immunoblotting of phosphoproteins and G3. Thus, the function of G3 may be further defined as involved in MAPKinase and cell cycle signal transduction pathways. A schematic pathway of the relationship of G3 to MAPKinase and cell cycle signaling is provided in FIG. 6.

The putative role of G3 relative to E2 and breast cancer cells appears to be in protection of the cell from apoptosis following estrogen stimulation. Early E2 signaling pathways can initiate an apoptotic cascade unless blocked to allow the cell to commit to cell cycle progression. The expression profile of G3 indicates that it can function to block apoptosis and allow the transformed cell to proceed through the cell cycle, even in the presence of other pro-apoptotic signals. Over expression of G3 through high doses of E2, would do just the opposite: the transformed cell is committed to apoptotic pathways. This is observed experimentally with high dose E2 treatment of MCF-7 cells.

Thus, controlling the level of expression of G3 or its kinase activity controls the viability of the breast cancer cell. Either blocking the expression of G3 or its kinase activity would be expected to have antineoplastic effects. Loss of control of G3 expression may also allow transformed cells to undergo uncontrolled cell growth. Similar inferences may be made for other hormonally responsive tissues such as endometrium.

### Example 2

### Construction of a Transgenic Animal

Characterization of E2IG3 genes provided information that is necessary for generation of E2IG3 transgenic animal models to be developed by homologous recombination (for knockouts) or transfection (for expression of E2IG3 fragments, antisense, AP RNA r increased expression of wild-type or mutant E2IG3s). Such models may be mammalian animal e.g. a mouse. These models are useful for the identification of cancer therapeutics alone or in combination with cancer inducing cells or agents, or when such mice are crossed with mice genetically predisposed to cancers.

The preferred transgenic animal has overexpression in E2IG3 and has predisposition to cancer. This mouse is particularly useful for the screening of potential cancer therapeutics.

### Example 3

### E2IG3 Protein Expression

E2IG3 genes and fragments thereof may be expressed in both prokaryotic and eukaryotic cell types. If an E2IG3 fragment modulates apoptosis by exacerbating it, it may be desirable to express that protein under control of an inducible promoter.

In general, E2IG3 fragments may be produced by transforming a stable host cell with all or part of the E2IG3-encoding cDNA fragment that has been placed into a suitable expression vector.

Those skilled in the art of molecular biology will understand that a wide variety of expression systems may be used to produce the recombinant protein. The precise host cell used is not critical to the invention, although cancer cells are preferable. The E2IG3 protein may be produced in a prokaryotic host (e.g. E. coli) or in a eukaryotic host (e.g. S. cerevisiae, in cells such as Sf21 cells, or mammalian cells such as COS-1, NIH3T3, or HeLa cells, or other highly proliferative cell types). These cells are publicly available, for example, from the American Type Culture Collection, Rockville, Md.; see also Ausbel et al., Current Protocols in Molecular Biology, John Wiley & Sons, New York, N.Y. 1994). The method of transduction and the choice of the expression vehicle will depend on the host system selected. Transformation and transfection methods are described, e.g. in Ausbel et al. (supra), and expression vehicles may be chosen from those provided, e.g. in Cloning Vectors: A Laboratory Manual (P. H. Pouwels et al., 1985, Supp. 1987).

Polypeptides of the invention, particularly short E2IG3 fragments, can also be produced by chemical synthesis (e.g. by the methods described in Solid Phase Peptide Synthesis, 2^{nd} ed., 1984, The Pierce Chemical Co., Rockford, Ill.). These general techniques of polypeptide expression and purification can also be used to produce and isolate useful E2IG3 fragments or analogs, as described herein.

### Example 4

### Anti E2IG3 Antibodies

In order to generate E2IG3-specific antibodies, an E2IG3 coding sequence (e.g. amino acids 180-276 c.f. FIG. 3) can be expressed as a C-terminal fusion with glutathione S-transferase (GST; Smith et al., Gene 67:31-40, 1988). The fusion protein can be purified on glutathione-Sepharose beads, eluted with glutathione, and cleaved with thrombin (at the engineered cleaved site), and purified to the degree required to successfully immunize rabbits. Primary immunizations can be carried out with Freund's compete adjuvant and subsequent immunizations performed with Freund's incomplete adjuvant. Antibody titers are monitored by Western blot and immunoprecipitation analysis using the thrombin-cleaved E2IG3 fragment of the GST-E2IG3 fusion protein. Immune sera are affinity-purified using CNBr-Sepharose-coupled E2IG3 protein. Antiserum specificity is determined using a panel of unrelated GST proteins (including (GST p53, Rb, HPV-16 E6, and E6-AP) and GST-trypsin (which was generated by PCR using known sequences).

As an alternate or adjunct immunogen to GST fusion proteins, peptides corresponding to relatively unique hydrophilic regions of E2IG3 may be generated and coupled to keyhole limpet hemocyanin (KLH) through an introduced C-terminal lysine. Antiserum to each of these peptides is similarly affinity purified on peptides conjugated to BSA, and specificity tested by ELISA and Western blotting using peptide conjugates, and by Western blotting and immunoprecipitation using E2IG3 expressed as a GST fusion protein.

Alternatively, monoclonal antibodies may be prepared using the E2IG3 proteins described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T-Cell Hybridomas, Elsevier, N.Y., 1981; Ausbel et al., supra). Once produced, monoclonal antibodies are also tested for specific E2IG3 recognition by Western blot or immunoprecipitation analysis (by the methods described in Ausbel et al., supra).

Antibodies that specifically recognize E2IG3s or fragments of E2IG3s, such as those described herein containing the nucleotide binding site domain are considered useful in the invention. They may, for example, be used in an immunoassay to monitor E2IG3 expression levels or to determine the subcellular location of an E2IG3 or E2IG3 treatment produced by a mammal. Antibodies that inhibit E2IG3 cleavage products may be especially useful in inducing apoptosis in cells undergoing undesirable cell proliferation.

Preferably, antibodies of the invention are produced using E2IG3 sequence that does not reside within highly conserved regions, and that appears likely to be antigenic, as analyzed by criteria such as those provided by the Peptide structure program (Genetics Computer Group Sequence Analysis Package), Program Manual for the GCG Package, Version 7, 1991) using the algorithm of Jameson and Wolf (CABIOS, 4:181, 1988). Specifically, these regions, which are found between BIR1 and BIR2 of all E2IG3s, are from amino acid 99 to amino acid 170 of hiap-1, from amino acid 123 to amino acid 184 of hiap-2, and from amino acid 116 to amino acid 133 of either xiap or m-xiap. These fragments can be generated by standard techniques, e.g., by the PCR, and cloned into the pGEX expression vector (Ausbel et al., supra). Fusion proteins are expressed in E. coli and purified using a glutathione agarose affinity matrix as described in Ausbel et al., (supra). In order to minimize the potential for obtaining antisera that non-specific, or exhibits low-affinity binding to E2IG3, two or three fusions are generated for each protein, and each fusion is injected into at least two rabbits. Antisera are raised by injections in series, preferably including at least three booster injections.

### Example 5

### Identification of Molecules That Modulate E2IG3 Protein Expression

E2IG3 cDNAs facilitate the identification of molecules that decrease E2IG3 expression or otherwise enhance apoptosis normally blocked by the E2IG3s. In one approach, candidate molecules are added, in varying concentration, to the culture medium of cells expressing, E2IG3 mRNA. E2IG3 expression is then measured, for example, by Northern blot analysis (Ausebel et al., supra) using an E2IG3 cDNA, or cDNA fragment, as a hybridization probe. The level of E2IG3 expression in the presence of the candidate molecule is compared to the level of E2IG3 expression in the absence of the candidate molecule all other factors (e.g., cell type and culture conditions) being equal.

The effect of candidate molecules in E2IG3-mediated apoptosis may, instead, be measured at the level of E2IG3 protein level of E2IG3 fragments using the general approach described above with standard protein detection techniques, such as Western blotting or immunoprecipitation with an E2IG3-specific antibody (for example, the E2IG3 antibodies described herein).

Compounds that modulate the level of E2IG3 may be purified, or substantially purified, or may be one component of a mixture of compounds such as an extract or supernatant obtained from cells (Ausubel et al., supra). In an assay of a mixture of compounds E2IG3 expression is tested against progressively smaller subsets of the compound pool (e.g. produced by standard purification techniques such as HPLC or FPLC) until a single compound or minimal number of effective compounds is demonstrated to modulate E2IG3 expression.

Compounds may also be screened for their ability to enhance E2IG3-mediated apoptosis. In this approach, the degree of apoptosis in the presence of a to candidate compared to the degree of apoptosis in its absence, under equivalent conditions. Again the screen may begin with a pool of candidate compounds from which one or more useful modulator compounds are isolated in a step-wise fashion. Apoptosis activity may be measured by any standard assay, for example, those described herein.

Another method for detecting compounds that modulate the activity of E2IG3s is to screen for compounds that interact physically with a given E2IG3 polypeptide. These compounds may be detected by adapting interaction trap expression systems known in the art. These systems detect protein interactions using a transcriptional activation assay and are generally described by Gyuris et al. (Cell 75:791-803, 1993) and Field et al (Nature 340:245-246, 1989), and are commercially available from Clontech (Palo Alto, Calif.). In addition, PCT Publication WO 95/28497 describes an interaction trap assay in which proteins involved in apoptosis, by virtue of their interaction with Bc1-2, are detected. A similar method may be used to identify protein and other compounds that interact with E2IG3s.

Compounds or molecules that function as modulators of E2IG3-mediated cell death may include peptide and non-peptide molecules such as those present in cell extracts, mammalian serum, or growth medium in which mammalian cells have been cultured.

What is claims is:

1. A polypeptide comprising an E2IG3 sequence or a substantially identical fragment thereof.

2. A polynucleotide comprising a sequence encoding an E2IG3 polypeptide or substantially identical a fragment thereof.

3. An expression vector comprising the polynucleotide of claim 2.

4. A transformed cell comprising the polynucleotide of claim 2.

5. A transformed cell comprising the expression vector of claim 3.

6. The transformed host cell of claim 4, wherein the host cell is prokaryotic.

7. The transformed host cell of claim 4, wherein the host cell is eukaryotic.

8. The transformed host cell of claim 4, wherein the host cell is a cancer cell.

9. A method for identification of E2IG3 polypeptide function comprising:
treating cells with estrogen;
determining polynucleotide expression patterns using a gene array;
determining the polypeptide phosphorylation expression patterns; and
correlating the polynucleotide and polypeptide phosphorylation expression patterns.

10. An antibody that binds to an E2IG3 polypeptide or a substantially identical fragment thereof.

11. The antibody of claim 10, wherein the antibody binds a nucleotide binding site domain of the E2IG3 polypeptide.

12. A method for identification of molecules that modulate E2IG3 expression comprising:
adding candidate molecules to the culture medium of cells expressing E2IG3 mRNA;
measuring E2IG3 expresssion; and
comparing the level of E2IG3 expression in the presence of the candidate molecules to the level of E2IG3 expression in the absence of the candidate molecules.

13. The method of claim 12, wherein E2IG3 expression is measured by E2IG3 polynucleotide expression.

14. The method of claim 12, wherein E2IG3 expression is measured by E2IG3 polypeptide expression.

15. A method for identification of molecules that modulate E2IG3-mediated apoptosis comprising:
adding candidate molecules to the culture medium of cells expressing E2IG3 mRNA;
measuring the degree of apoptosis; and
comparing the degree of apoptosis in the presence of the candidate molecules to the degree of apoptosis in the absence of the candidate molecules.

16. A method of treating or preventing a proliferative disease in a mammal comprising administering a polypeptide of claim 1.

17. A method of treating or preventing a proliferative disease in a mammal comprising administering a polynucleotide of claim 2.

18. A method of treating or preventing a proliferative disease in a mammal comprising administering an antibody of claim 10.
